(19) United States
(12) Patent Application Publication
(10) Pub. No.: US 2022/0134079 A1
(43) Pub. Date: May 5, 2022

(54) ANTISEPTIC CONNECTOR COVER AND METHOD OF USE

(71) Applicants: Michael Wallace Howlett, Las Vegas, NV (US); Brad Charles Holt, Midvale, UT (US); James Victor Mercer, West Jordan, UT (US)

(72) Inventors: Michael Wallace Howlett, Las Vegas, NV (US); Brad Charles Holt, Midvale, UT (US); James Victor Mercer, West Jordan, UT (US)

(21) Appl. No.: 17/519,295

(22) Filed: Nov. 4, 2021

Related U.S. Application Data
(60) Provisional application No. 63/109,922, filed on Nov. 5, 2020.

(51) Int. Cl. A61M 39/16; A61M 39/10; A61M 39/20

(52) U.S. Cl. CPC A61M 39/162; A61M 39/20; A61M 39/1011

(57) ABSTRACT

A fluid-containing cap that fits onto corresponding tubing connectors comprised of a housing and rigid, non-compressible internal fluid reservoir. Fluid is retained within the cap by means of a high surface area internal structure providing sufficient surface tension to prevent fluid from exiting the reservoir until a corresponding tubing connector is inserted therein causing the fluid to disperse out of the cap reservoir and onto the inserted tubing connector.

### PRIORITY

The present application claims priority to and the benefit of U.S. PROVISIONAL PATENT APPLICATION SER. NO. 63/109,922 entitled “ANTISEPTIC CONNECTOR COVER AND METHOD OF USE” filed Nov. 5, 2020, the entirety of which is incorporated herein by reference.

### BACKGROUND

Medical connector covers and caps (e.g., red caps) have been in wide use in health care and particularly IV therapy for many decades. In the last ten years, connector covers containing isopropyl alcohol (IPA) and other antiseptics have been shown to help reduce the risk of infection and are now in extensive use. Antiseptic connector covers currently on the market utilize an internal absorbent sponge or similar absorbent material to retain the antiseptic fluid and then distribute or release this fluid onto the target connector when the connector cover is attached to the target connector.

Needleless injection sites (NIS) are a typical application for antiseptic connector covers, where when the NIS is inserted into the connector cover, the antiseptic or isopropyl alcohol is dispersed over the NIS fitting in order to provide a safe and sterile environment when the site is used for subsequent injections or treatments.

Recently some medical reports have called into question the safety of using an absorbent sponge to retain antiseptic fluid in these connector covers. Some have asserted that particles of the absorbent material or sponge may be sheared off during interface with the rigid plastic features (e.g., Luer threads) of an NIS or other target connector, thus creating the risk of these particles entering the patient's body through the tubing (embolization). Others have asserted that long term alcohol exposure could result in degradation of the absorbent material that could lead to harmful residue, such as sponge micro-particles or a chemical leachate, which could, in turn, enter the tubing and patient's body. Furthermore, antiseptic fluid, commonly 70% isopropyl alcohol (IPA), often drips out of the caps after opening their packaging with even more dripping occurring when the connector cover internal sponge is depressed upon application to the target connector such as NIS. This dripping is objectionable for at least the following reasons; it exposes the user's fingers to IPA, causing skin irritation; second, it creates an objectionable alcohol odor for both staff and patient; third, the IPA may get on unintended surfaces, potentially causing damage; fourth, the dripping IPA makes the cap slippery and more difficult to handle; and fifth, the vaporizing IPA is a fire hazard. The Bariatric Nurses Society recently published guidelines to eliminate IPA cap use during high-oxygen bariatric procedures. Additionally, as cap sponges dry out, they may act to draw fluid out of an NIS and potentially cause IV-line reflux. Dry sponges will also tend to pull environmental fluid into the cap. For example, a dry cap may absorb, or “suck up”, body fluids or other fluid that come into contact with the connector cover.

In summary, the deficiencies of current IPA connector covers containing a sponge are as follows; sponge may be damaged by threads of the fitting causing particle embolization risk, the sponge may degrade with IPA exposure, generating micro-particles or a chemical leachate, IPA dripping from the connector cover causing needless user skin exposure, especially to the fingers of the user, IPA dripping from connector cover creates objectionable odor, IPA dripping from the connector cover may drip onto and damage items it is not intended for, such as nearby medical equipment, or patient personal items, IPA dripping from connector cover makes it slippery (“watermelon seed effect”) and more difficult to handle, IPA vaporization can present a fire risk, and, a dry connector cover sponge may absorb unwanted fluids if exposed.

Yet another significant consideration is production cost; eliminating a sponge during manufacture of the connector cover eliminates the sponge material, as well, as the manufacturing steps of cutting and inserting the sponge material into the cover.

What is needed is an antiseptic connector cover which presents the advantage of providing isopropyl alcohol to sterilize reusable medical connections, such as, an NIS, IV or Luer type fitting, without a compressible material such as a soft sponge which allegedly can be compromised during engagement of a threaded connector cover or by degradation following prolonged exposure to IPA.

### SUMMARY OF THE INVENTION

The disclosure of the present invention relates to a medical fitting capping device or more specifically, to a sponge-less antiseptic connector cover for medical fittings such as NIS, IV or Luer. The device of the present invention herein described addresses the above problems by eliminating a compressible sponge or absorbent material, replacing it with a unique mechanism for retaining a sterile fluid, commonly 70% isopropyl alcohol (IPA), in a sponge-less, rigid reservoir by means of surface tension. While the present disclosure will refer to the use of IPA in the reservoir, it should be understood that other sterile fluids and/or antiseptic fluids may be used. Substantially no IPA will drip out of the device even when it is inverted with the open end facing downward. Further, once the device is applied to the target NIS and the forward surface of the NIS contacts and abuts the top of the fluid retention structure, IPA will be distributed onto the NIS to disinfect it.

Although there are myriad applications for this device, including non-medical ones, such as, lubrication of industrial air control fittings and sterilization of food processing couplings, the primary purpose of the invention is to provide an antiseptic connector cover for NIS and IV connectors that disinfects and protects, and does this without a sponge and without messy out-dripping when handling.

### Objects of the Invention

1. Elimination of a sponge or absorbent material as a fluid retention device to prevent fluid from dripping out of an open-ended fluid reservoir within a cover/cap when it is inverted. This is accomplished by increasing the surface area within the fluid reservoir by means of fins, projections or similar construction that create narrow spaces or channels where fluid resides. In some examples, a textured surface, such as a rough surface or grooves in the surface may increase surface area and fluid adherence to the surface area. Fluid adhering to the multiple surfaces within the cap will retard fluid outflow.

2. Dripless containment of fluid within an open-ended sponge-less reservoir, even when it is inverted with the open end facing downward.

3. Providing for communication or distribution of fluid within the reservoir onto an inserted implement such as a connector.

4. The removable connector cover covers and protects the implement to prevent touch and environmental contamination.

In a first embodiment of the present invention, an antiseptic medical connector cover comprises an extended body structure including a rigid internal antiseptic reservoir, a fluid retention structure inside of the reservoir, a reservoir face, followed by an connection portion including a mechanical interlock feature configured to engage a reciprocal medical fitting such as NIS, IV or other Luer type fitting. For example, the mechanical interlock feature may include internal threads, detents or recesses for a snap fit, channels to allow a twist lock (similar to a BNC connector), or other mechanisms by which the cover can connect to and be retained on a medical connector. An outer surface of the extended body structure can be formed as a cylinder or having a polygonal cross-section in order to provide a secure gripping shape and surface. IPA may be distributed onto the target connector to disinfect it one or both of two mechanisms: first, the insertion of the target medical connector will displace IPA volume towards the opening of the cap. Secondly, IPA will be drawn out of the fluid retention structure and onto the face and threads of the target medical connector by the force of surface tension or capillary action.

In some embodiments, the fluid retention structure is a compressible structure. In other embodiments, the fluid retention structure is a non-compressible structure. For example, a compressible structure is any structure that deforms under 10 Newtons or less of longitudinal force. In some embodiments, the fluid retention structure deforms elastically under 10 Newtons or less of longitudinal force. In some embodiments, the fluid retention structure deforms plastically under 10 Newtons or less of longitudinal force.

In one embodiment, the fluid retention structure includes a plurality of fins configured in a radial pattern extending from the inside surface of the antiseptic reservoir toward the center; a first plurality of fins in the pattern, being longer, extending substantially from the inside surface toward a center line of the rigid antiseptic reservoir, and a second plurality of fins is shorter. In some embodiments, the short second plurality of fins extends from the inside surface to approximately one half of the distance from the inside surface of the reservoir to the center line. The short fins reside within a triangular void formed between adjacent long fins from the first plurality of fins. Spacing between each of the fins within the full plurality is such that voids between the fins are wide enough to retain an maximum amount of IPA yet close enough that the surface tension and molecular attraction between IPA molecules and the fin structures causing the IPA to stay retained within the rigid reservoir until at least a portion of the IPA is contacted by the target medical fitting such as a NIS or Luer fitting.

In yet another embodiment of the present invention, the ridged internal antiseptic reservoir again includes a plurality of alternating long and short fins formed in radial pattern as described above, further including a long fin including a rod or cylinder formed integrally with the free end of the fin. The cylinder may be formed substantially on the centerline of the internal reservoir and provides additional contact surface area proximate a central void formed at ends of the plurality of long fins. It is understood that the additional contact surface area will provide additional molecular attachment points for the IPA and more secure retention due to molecular attraction.

In other embodiments the plurality of fins formed in radial pattern within the body of the antiseptic reservoir may include fins having uniform folds of waves which match or mirror adjacent fins. In another embodiment the plurality of fins may include a radial pattern of fin where the fins incorporate one or more cross-members in order to preserve proper spacing between the fins. For example, the adjacent surfaces of the fluid retention structures (e.g., fins, rods, cross-members) may be positioned and arranged to follow one another approximately complementarily at a substantially constant spacing. In at least one example, the spacing between the features of the fluid retention structures is between 0.25 millimeters (mm) and 2.0 mm. In a specific example, the spacing between the fluid retention structures is between 0.75 mm and 1.25 mm.

It is contemplated that each of the above-described embodiments may be printed or injection molded as a fully integral structure without any additional assembly or manufacturing steps beyond filling with a desired fluid, such as IPA, sealing, and packaging.

Another embodiment of the antiseptic connector cover of the present invention, the rigid internal reservoir includes a plurality of rods or columns formed as a bundle having at least one attached end. The rod spacing within the bundle such that an appropriate amount of IPA can be retained within the rigid antiseptic reservoir but again the rods must be close enough that molecular attraction and resulting surface tension in the IPA will cause that the IPA is retained within the reservoir. In one embodiment the rods are cylindrical having a circular cross-section, wherein, other possible embodiments the cross section may be one of oval, square, rectangular, triangular or another polygonal shape. In a similar embodiment the plurality of rods or columns may be described as a bundle of brush fibers or other similar fine filaments. It is contemplated the that the bundle of rods or columns can be injection molded as a separate component or formed using a fine synthetic material that is bundled and cut to an appropriate length prior to insertion into a cavity formed in the body housing.

In another embodiment the rigid internal reservoir may include a plurality of tubes in lieu of rods or may consist of a plurality of holes drilled or formed inside of the antiseptic connector cover.

In one embodiment the extended cap body has an internal reservoir configured to receive a collapsible fluid retention structure comprising a plurality of the folded fins formed in a radial pattern. Sufficient space is provided within the plurality of fins in order to provide an adequate quantity of an antiseptic fluid such as, IPA. It is contemplated that during manufacturing the top portion of the fluid retention structure will extend into the threaded portion of the internal reservoir and the fluid retention structure will be pressed further into the fluid reservoir or is compressed when it is engaged by a compatible threaded fitting. Compression of the folded fins will further displace the antiseptic fluid onto the reciprocal fitting.

In yet another embodiment a rigid ridged internal reservoir element may be formed using a sheet or thin film material having at least one textured or shaped surface. The film may be cut to an appropriate width and length prior to be rolled into a cylinder that is inserted into the reservoir cavity formed in the body housing. Any texture providing enough relief between the rolled layers of the film material in order to trap, a fluid, or the IPA, may be formed on at least one surface of the film. In one embodiment a series of linear ridges and valleys, or a plurality of grooves or channels may be formed in the film. The film is then cut perpendicular to the plurality of channels where when the film is rolled into a cylinder, the channels remain open between layers and extend through the cylinder parallel with the long central axis of the cylinder.

And in another embodiment a fluid retention screen is formed on the open face of the rigid internal reservoir. The fluid retention screen having a plurality of small openings for the distribution of the IPA retained within the reservoir onto a reciprocal threaded fitting. In one embodiment the openings are configured as a plurality of slits formed in a radial pattern. In another embodiment the openings may be a series of holes formed randomly or in a pattern. The hole can be circular or any desired shape.

In some embodiments, a surface of at least one fluid retention feature of the fluid retention structure includes a textured surface. For example, a rough surface or grooves in the surface may increase surface area and fluid adherence or cohesion to the surface of the fluid retention feature.

In the above-described embodiments, the reservoir is closed having a integrally formed closed bottom and it is understood that in order to fill such a reservoir completely without forming air pockets or for providing a consistent IPA fill quantity, it may be beneficial to fill from the bottom of the reservoir up. In some embodiments, the reservoir may be filled from the top down or through ports in the sidewalls or ends of the cap. It is contemplated that the rigid reservoir could be filled using a blunt hollow needle or tube to inject the appropriate amount of IPA before the open threaded end of the connector cover or cap is sealed with an adhesive foil or plastic film and then placed into a sterile package. In some embodiments, vibrating or shaking the filled reservoir may release any bubbles or vapor trapped in the liquid in the reservoir before sealing the filled reservoir. In some embodiments, additional liquid may be inserted into the reservoir after vibration to ensure the reservoir is completely filled.

In some embodiments, the IPA or other sterile fluid is inserted into the internal reservoir to cover the fluid retention structure. For example, the IPA or other sterile fluid may be inserted into the internal reservoir to fill the internal reservoir until a meniscus of the IPA or other sterile fluid is above the fluid retention structure. In at least one example, the IPA or other sterile fluid may be inserted into the internal reservoir to fill the internal reservoir until a meniscus of the IPA or other sterile fluid is above the fluid retention structure.

Other embodiments of the present invention may include a reservoir having an open bottom as well as open threaded end or connecting end. In such an embodiment the open end or threaded end may be sealed using an adhesive film prior to a proper amount of IPA being added directly into the open reservoir bottom. It is contemplated that the void formed in the threaded end will provide enough air relief to allow the IPA to flow into the rigid reservoir structure without forming any excessive air pockets or voids. Once the proper amount of IPA is added to the reservoir an additional adhesive film can be attached to fully seal the antiseptic connector cover prior to packaging.

In one embodiment a recess may be formed where the bottom adhesive film is attached, the recess will allow the film to be attached but will protect the bottom film from being inadvertently removed during use and will protect the film by not allowing a user to scrap the film edge loose using a fingernail or another tool used for scraping.

Yet another embodiment of the present invention is to provide a concave cap configured to form a recessed fit into the threaded portion of the internal fluid reservoir. The concave cap formed using a sterile elastomer material and configured to make an interference or compression fit into the threaded opening. It is understood that the concave portion of the cap may keep any antiseptic fluid away from the threaded portion of the fluid reservoir and force the fluid into the fluid retention structure. The concave cap may include a pull tab configured for easy cap removal.

### BRIEF DESCRIPTION OF THE DRAWINGS

The following description of the embodiments can be understood in light of the Figures, which illustrate specific aspects of the embodiments and are part of the specification. Together with the following description, the Figures demonstrate and explain the principles of the embodiments.

FIG. 1 A front perspective view of an antiseptic medical connector cover,

FIG. 2 a top end view of a connector cover,

FIG. 3 a top end view of a connector cover having a fluid retention screen,

FIG. 4 a bottom end view of an open rigid reservoir,

FIG. 5 a front perspective view of a connector cover with rod retention elements,

FIG. 6 a front perspective view of a connector cover with cross retention elements,

FIG. 7 a front perspective view of a connector cover with vane retention elements,

FIGS. 8A, 8B and 8C is one embodiment of a fluid retention element with folded fins,

FIG. 9 is one embodiment of the concave cap, and,

FIGS. 10A and 10B are yet another embodiment where the fluid retention element is a textured rolled film.

### DETAILED DESCRIPTION

The following description of the embodiments can be understood in light of the Figures which illustrate specific aspects of the embodiments and are part of the specification. Together with the following description, the Figures demonstrate and explain the principles of the embodiments. In the Figures the physical dimensions of the embodiment may be exaggerated for clarity. The same reference numerals or word descriptions in different drawings represent the same element, and thus their descriptions may be omitted.

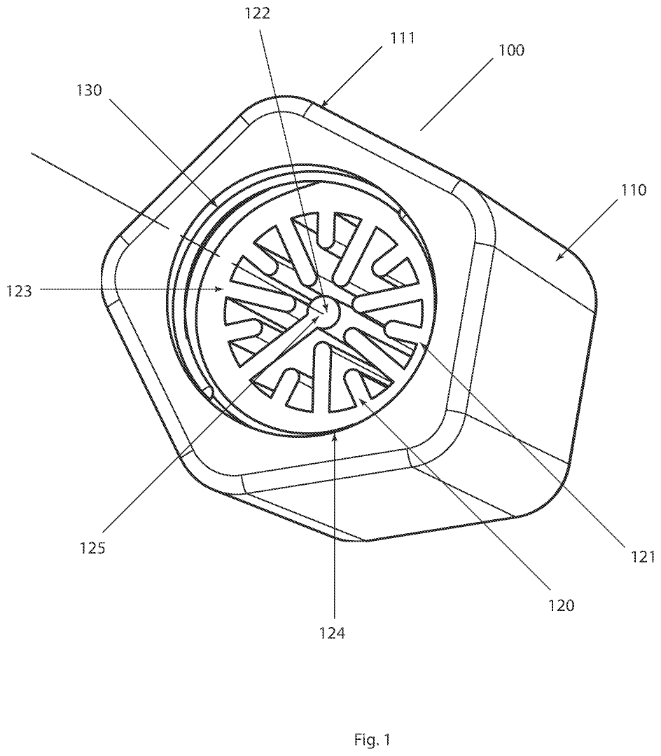

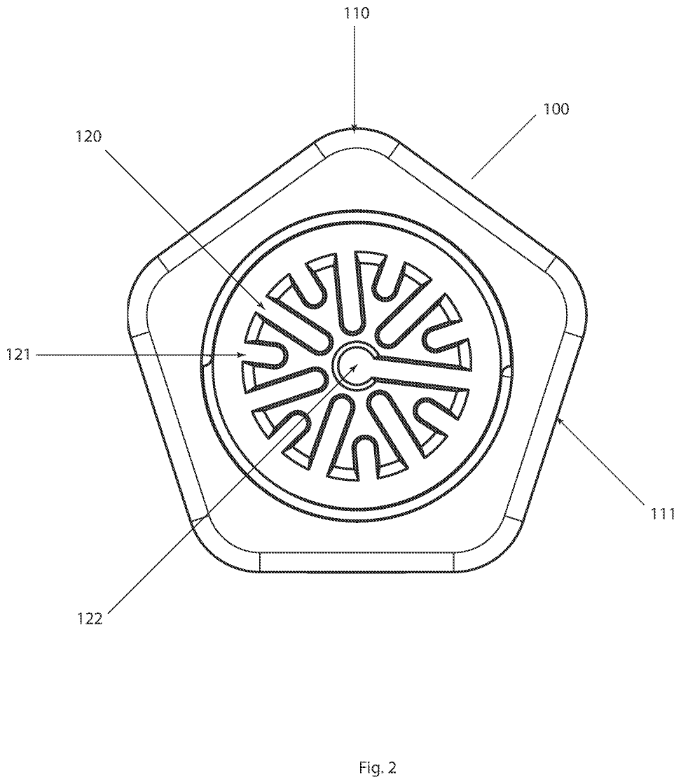

A first embodiment of the present invention or Antiseptic Medical Connector Cover 100, as described in FIGS. 1 and 2, including an extended body portion 110 comprising a rigid reservoir 120 having fluid retention structure 121. The reservoir formed in the bottom portion of the body 110 with a connector receiver 130 formed in the top portion, the connector receiver having an internal thread 131 configured to fit onto a typical medical fitting such as NIS or a Luer type fitting. The extended body portion 110 may be cylindrical having a round cross-section or may be described as columnar having a polygonal cross-section which may be one of square, triangular, or another polygon. The outside surface 111 including such formed facets in order to provide a secure gripping surface.

In one embodiment the fluid retention structure 121 includes a plurality of fins formed in a radial pattern, a first of the plurality of fins being longer, extending from the inside reservoir surface 124 to proximate the center line 124 of the extended body portion 111. A second plurality of short fins dispersed between the plurality of long fins. In yet another embodiment, a central spacing rod 122 is attached at the end of one of the plurality of long fins.

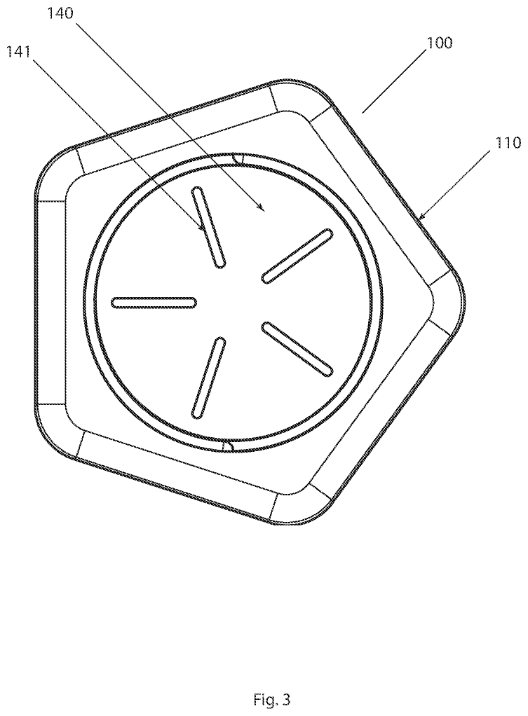

FIG. 3 describes an embodiment of the present invention where a fluid retention screen 140 is formed at the free end of the rigid reservoir 120. The fluid retention screen 140 may include a plurality of openings or slits 141 designed to control the release of IPA onto a reciprocal medical connector. While the present disclosure will refer to the use of IPA in the reservoir, it should be understood that other sterile fluids (such as sterile water) and/or antiseptic fluids may be used.

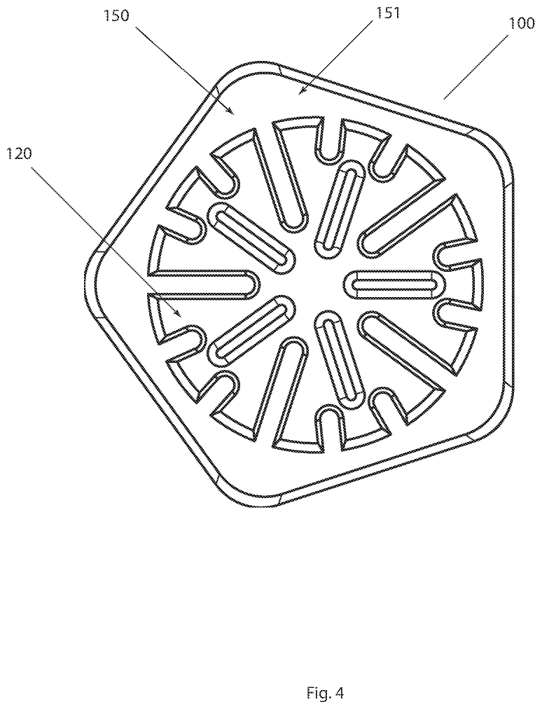

The embodiment of FIG. 4 of the present invention may include a rigid reservoir 120 having an open bottom 150 as well as the open threaded end or connecting end. In one embodiment of FIG. 4, a recess 151 may be formed where a bottom adhesive film is attached.

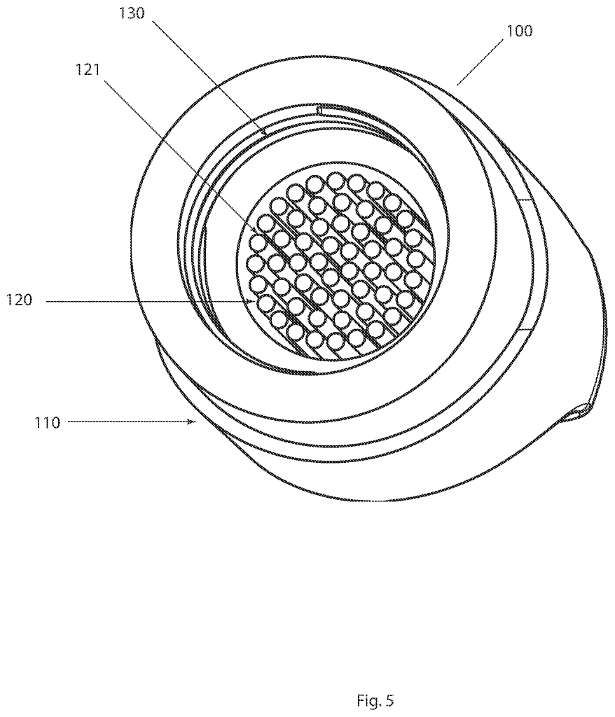

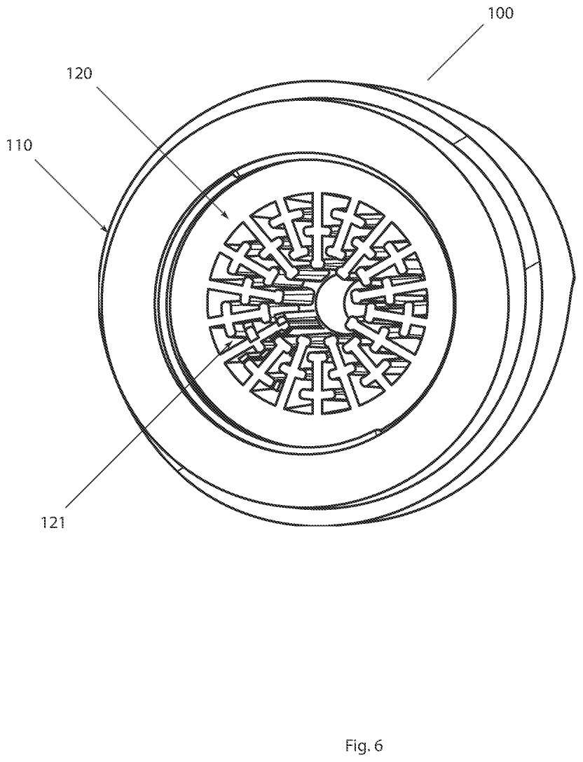

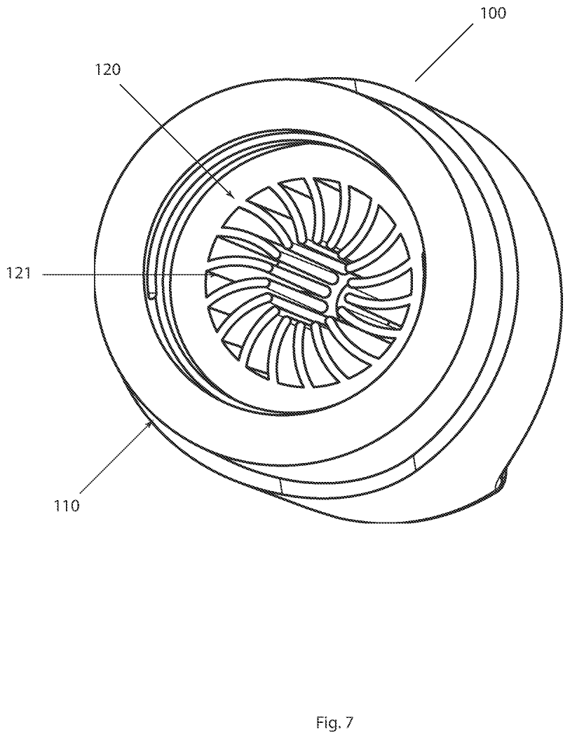

In yet other embodiment of the present invention or Antiseptic Medical Connector Cover 100, the rigid reservoir 120 may be formed using alternate fluid retention structures 121, such as the plurality of curved vanes shown in FIG. 7 or the interlocking cross shaped fins of FIG. 6. Whereas FIG. 5 depicts an embodiment where the fluid retention structure 121 is formed using a plurality of rods formed as a bundle and attached at one end. In some embodiments, spacing the fluid retention elements within the fluid retention structure 121 at such distances in order to increase IPA capacity while having the fixtures close enough to one another where the surface tension within the IPA will secure the IPA within the fluid retention structure 121 even when the connection cover is inverted and moved about may be beneficial.

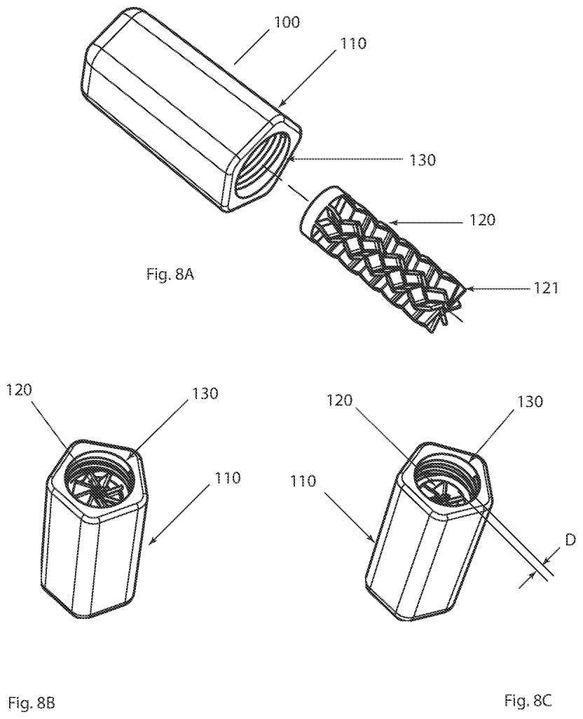

In the embodiment of FIGS. 8A through 8C the connection cover 100 is formed where the body portion 110 includes an empty central void and a threaded opening which are configured to receive a reservoir insert 120. The fluid retention structure 120 may include a plurality folded fins 121 formed in a radial pattern that can be compressed when the cover 100 is placed on a reciprocal fitting. FIG. 8B shows the fluid retention structure 120 extending proximate the bottom of the threaded portion 130, whereas, as shown in FIG. 8C the fluid retention structure 120 is compressed longitudinally further into the body portion 110 by a distance D. The plurality of folded fins 121 will compress in an accordion fashion and release the retained IPA when displaced by a fitting such as a NIS, IV or Luer.

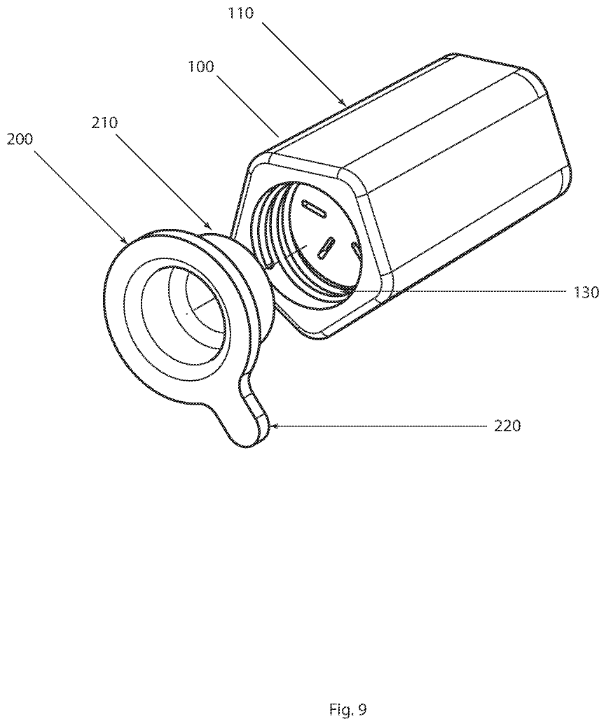

FIG. 9 is an embodiment of the present invention or antiseptic connector cover 100 having a concave protective cap 200. The connector cover 100 comprises an extended body portion 110 including an internal reservoir having a threaded opening 130. The cap 200 has a concave portion or nipple 210 that extends down and is configured to form a secure interference fit within the threaded opening 130. The concave portion 210 is configured to force antiseptic fluid to stay within the fluid retention structure. A pull tab 220 may be used for easy removal of the concave protective cap 200.

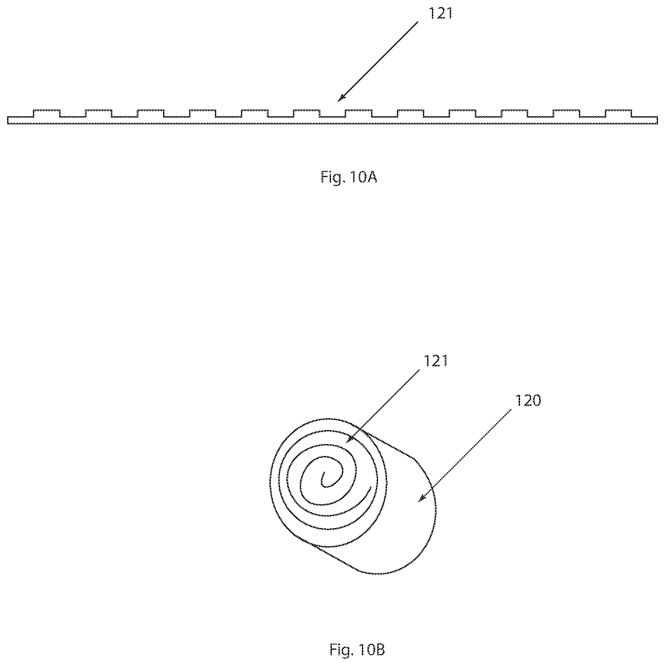

The embodiment disclosed in FIGS. 10A and 10B includes a thin film material used to form a fluid retention structure 121 having surface texturing or a plurality of fluid retention elements including a repeating pattern of ridges and grooves. The thin film may be rolled into a small cylinder in order to form a reservoir insert 120, which may be placed into the void formed body portion or housing 110 as shown in FIG. 8.

It is to be understood that the above-mentioned arrangements are only illustrative of the application of the principles of the present disclosure. Numerous modifications or alternative arrangements may be devised by those skilled in the art without departing from the spirit and scope of the present disclosure and the appended claims are intended to cover such modifications and arrangements. Thus, while the present disclosure has been shown in the drawings and described above with particularity and detail, it will be apparent to those of ordinary skill in the art that numerous modifications, including, but not limited to, variations in size, materials, shape, form, function and manner of operation, assembly and use may be made without departing from the principles and concepts set forth herein.

We claim:

1. A medical connector cover comprising,
an extended body having an open end and a closed end;
an internal reservoir in the extended body at the open end, the internal reservoir including at least one fluid retention structure,
a connector receiver; and
the connector receiver including a mechanical interlock.

2. The connector cover of claim 1, wherein the fluid retention structure is an integral part of the connector cover.

3. The connector cover of claim 1, wherein the fluid retention structure is formed as a removable insert.

4. The connector cover of claim 3, wherein the fluid retention structure includes a plurality of fins formed in a radial pattern.

5. The connector cover of claim 3, wherein the fluid retention structure is compressible in a longitudinal direction of the extended body.

6. The connector cover of claim 1, wherein the fluid retention structure is non-compressible.

7. The connector cover of claim 1, wherein the fluid retention structure includes a plurality of fluid retention elements.

8. The connector cover of claim 7, wherein at least one fluid retention element is a fin.

9. The connector cover of claim 7, wherein at least one fluid retention element is a rod.

10. The connector cover of claim 1, wherein the fluid retention elements retain IPA therebetween when the connector cover is inverted with the open end downward.

11. The connector cover of claim 1, wherein the mechanical interlock includes internal threads.

12. The connector cover of claim 1, further including a sealing component that seals the open end of the extended body.

13. The connector cover of claim 12, wherein at least a portion of the sealing component protrudes into the internal reservoir.

14. The connector cover of claim 12, wherein the sealing component is coupled to the open end of the extended body by a compression fit.

15. The connector cover of claim 12, wherein the sealing component is coupled to the open end of the extended body by engagement with the mechanical interlock.

16. A system of protecting a medical connector, the system comprising:
a medical connector including a protruding end with a first mechanical interlock;
a connector cover including a second mechanical interlock, the connector cover coupled to the medical connector by the first mechanical interlock engaged with the second mechanical interlock, the connector cover including:
an extended body having an open end and a closed end,
an internal reservoir in the extended body at the open end,
the internal reservoir including at least one fluid retention structure, a connector receiver, and,
the connector receiver including the second mechanical interlock; and
an antiseptic fluid contained in the internal reservoir.

17. The system of claim 16, wherein the antiseptic fluid is IPA.

18. The system of claim 16, wherein the first mechanical interlock and second mechanical interlock are a Luer lock.

19. A method of filling an antiseptic medical connector cover, the method comprising:
positioning an antiseptic fluid in a connector cover, the connector cover including:
an extended body having an open end and a closed end,
an internal reservoir in the extended body at the open end, the internal reservoir including at least one fluid retention structure,
a connector receiver, and,
the connector receiver including a mechanical interlock,
wherein positioning the antiseptic fluid in the connector cover includes filling the internal reservoir from a bottom up to cover the at least one fluid retention structure.

20. The method of claim 19, further comprising:
vibrating the connector cover to release at least one vapor bubble from the antiseptic fluid; and
filling a remaining volume of the internal reservoir with additional antiseptic fluid.
